(19) United States
(12) Patent Application Publication
(10) Pub. No.: US 2026/0171219 A1
(43) Pub. Date: Jun. 18, 2026

(54) METHODS AND SYSTEMS FOR PROVIDING SUBSEQUENT SCAN DATA BASED ON SCAN GEOMETRY DATA

(71) Applicant: Siemens Healthineers AG, Forchheim (DE)

(72) Inventors: Fabian WAGNER, Erlangen (DE); Rainer SCHNEIDER, Hoechstadt (DE); Vanya SAKSENA, Nuernberg (DE); Dominik PAUL, Bubenreuth (DE)

(73) Assignee: Siemens Healthineers AG, Forchheim (DE)

(21) Appl. No.: 19/421,900

(22) Filed: Dec. 16, 2025

(30) Foreign Application Priority Data
Dec. 17, 2024 (DE) 10 2024 212 034.0

(51) Int. Cl. G16H 30/20; G06N 20/00; G16H 30/40

(52) U.S. Cl. CPC G16H 30/20; G06N 20/00; G16H 30/40

(57) ABSTRACT

A computer-implemented method comprises: obtaining first scan data of a subject from a medical imaging system; providing a trained machine learning model configured to determine, based on the first scan data, scan geometry data for a subsequent scan; applying the trained machine learning model to the first scan data to obtain scan geometry data for a subsequent scan of the subject; obtaining, from the medical imaging system, the subsequent scan data of the subject using the scan geometry data; and providing the subsequent scan data.

### CROSS-REFERENCE TO RELATED APPLICATION(S)

The present application claims priority under 35 U.S.C. § 119 to German Patent Application No. 10 2024 212 034.0, filed Dec. 17, 2024, the entire contents of which are incorporated herein by reference.

### FIELD

Aspects of one or more example embodiments of the present invention relate to methods and systems for providing subsequent scan data. In particular, aspects of one or more example embodiments of the present invention relate to systems and methods for deriving scan geometry data from first scan data. Further, aspects of one or more example embodiments of the present invention relate to the training of a trained machine learning model for providing scan geometry data for a subsequent medical scan.

### BACKGROUND

Automation of workflows is still a challenge in the acquiring and processing of medical images for arriving at a possible diagnosis of the patient. Thereby, medical imaging starts with tasks which might appear trivial at first sight such as the setting of suitable imaging parameters or the selection of an appropriate field of view (FOV). Typical workflows continue after imaging with post-processing tasks such as segmentation, in particular with computer aided detection (CAD) tools, or image quality assessments.

Automating these and other steps is difficult as these steps crucially depend on the underlying medical image. To put it differently, different diagnoses and tasks within the medical workflow may require different medical data with different imaging parameters. The step of defining the imaging parameters and/or the field of view is there for a crucial task, that still is often conducted by medical staff and/or technicians manually.

Typically, medical technicians conduct a first scan, roughly positioning the patient based on external anatomical markers. This positioning is followed by a low-resolution first (localizer) scan to determine the precise FOV and scan geometry for a clinical scan. The scan geometry, which includes slice alignment, specifies the angle and direction in which the anatomy of interest is imaged, influencing the radiologist's ability to make accurate diagnostic decisions from the tomographic slices displayed on 2D screens and various other post-processing tasks.

The manual determination of scan alignment based on the first (localizer) image is a time-consuming and error-prone task performed manually by medical technicians. This introduces variability and potential inaccuracies in the anatomical views used for diagnosis. What is more, staff shortages may lead to additional time pressure in the clinical routine.

### SUMMARY

While there are first methods for automizing the imaging workflow, the inventors have found that the accuracy for correctly predicting scan geometry data in particular is not sufficient. Further, suchlike methods systematically do not utilize information available about future processing of the image data in further steps of the workflow.

Accordingly, it is an object of embodiments of the present invention to improve slice alignment planning for the acquisition of medical images. In particular, it is an object of embodiments of the present invention to improve the prediction of scan geometry data for the image acquisition of subsequent images. Furthermore, it is an object of embodiments of the present invention to, in particular, automatically provide scan geometry data optimized for subsequent steps in the medical diagnosis workflow.

To solve at least this object a computer-implemented method for providing subsequent scan data of a subject, a computer-implemented method for providing a trained machine learning model, a medical imaging system, corresponding computer-program products and computer-readable storage media according to the main claims is proposed.

Alternative and/or preferred embodiments are object of the dependent claims.

In the following, the technical solution according to the present invention is described with respect to the claimed apparatuses as well as with respect to the claimed methods. Features, advantages, or alternative embodiments described herein can likewise be assigned to other claimed objects and vice versa. In other words, claims addressing the inventive method can be improved by features described or claimed with respect to the apparatuses. In this case, e.g., functional features of the method are embodied by objective units or elements of the apparatus.

The technical solution will be described both with regard to methods and systems for providing an updated machine learned function and also with regard to methods and systems for providing training or test data for updating a machine learned system. Features and alternate forms of embodiments of data structures and/or functions for methods and systems for providing machine learned functions can be transferred to analogous data structures and/or functions for methods and systems for providing training or test data. Analogous data structures can, in particular, be identified by using the prefix “training”. Furthermore, the prediction functions used in methods and system for providing information can, in particular, have been adjusted and/or trained and/or provided by methods and systems for adjustment of prediction functions.

According to one aspect, a computer-implemented method for providing subsequent scan data of a subject is provided. First scan data of the subject is obtained from a medical imaging system. A trained machine learning model configured to determine, based on first scan data, scan geometry data for a subsequent scan is provided. The trained machine learning model is applied to the first scan data to obtain scan geometry data for a subsequent scan of the subject. subsequent scan data is obtained, from the imaging system, of the subject using the scan geometry data. The subsequent scan data is provided.

The scan geometry data may in particular comprise slice alignment data.

The first scan data may comprise medical image data and/or non-image data. The subsequent scan data may comprise medical image data and/or non-image data. The first scan data and subsequent scan data may differ in their properties, for example, in contrast, resolution and/or size. The non-image data may comprise a scan protocol and/or scan documentations for example. The medical image data may comprise a two-dimensional image. Further, the medical image data may comprise a three-dimensional image. Further, the medical image data may comprise a four-dimensional image, where there are three spatial and one time-like dimensions. Further, the first scan data and subsequent scan data may comprise a plurality of individual medical images, in particular a medical image data set.

Medical image data may comprise image data, for example, in the form of a two- or three-dimensional array of pixels or voxels. Such arrays of pixels or voxels may be representative of color, intensity, absorption or other parameters as a function of two or three-dimensional position, and may, for example, be obtained by suitable processing of measurement signals obtained by a medical imaging modality and/or image scanning facility. Medical image data may depict a body part of a patient. Accordingly, it may contain two or three-dimensional image data of the patient's body part. The medical image data may be representative of an image volume and/or a cross-section through the image volume. The patient's body part may be comprised in the image volume. Medical image data may comprise data of a plurality of image slices. The slices respectively may show a cross-sectional view of the image volume. The arrangement of slices in the medical image data set may be determined by the imaging system (also modality) or by any post-processing scheme, function and/or method used. Further, image slices may artificially be defined in the imaging volume spanned by the medical image data set. In particular, this may happen as a function of the image data comprised in the first scan data in order to optimally pre-process the medical image data set for the ensuing diagnostic workflow.

The medical image data may be stored in a standard image format such as the Digital Imaging and Communications in Medicine (DICOM) format and in a memory or computer storage system such as a Picture Archiving and Communication System (PACS), a Radiology Information System (RIS), and the like. Whenever DICOM is mentioned herein, it shall be understood that this refers to the “Digital Imaging and Communications in Medicine” (DICOM) standard, for example according to the DICOM PS3.1 2020c standard (or any later or earlier version of said standard). The PACS may store the medical images in a post-processed version after acquisition. The post-processed medical image data may relate to readily viewable version in an image viewer and not to raw data.

A medical imaging system may correspond to a system used to generate or produce medical image data. For example, a medical imaging modality may be a computed tomography system (CT system), a magnetic resonance system (MR system), an angiography (or C-arm X-ray) system, a positron-emission tomography system (PET system) or the like. Specifically, magnetic resonance systems are a widely used and known medical imaging systems. Magnetic Resonance Imaging (MRI), to provide one example, is an advanced medical imaging technique which makes use of the effect magnetic field impacts on movements of protons. In MRI systems, the detectors are antennas, and the signals are analyzed by a computer creating detailed images of the internal structures in any section of the human body.

The scan geometry data, also slice alignment data, may specify the angle and/or direction, in which an anatomy of interest and/or a region of interest is imaged. The anatomy of interest may comprise for example an organ, a lesion and/or other parts of a human body. In particular, the scan geometry may be determined in a way that allows the radiologist to later make a diagnostic decision based on familiar anatomy views and/or to use post-processing functions and/or methods on the subsequent scan data. The scan geometry data may relate to an image acquisition procedure, in particular a scan, with a medical imaging system and/or modality with which the medical image, in particular the subsequent scan data, has been acquired. The scan geometry data may comprise a defined data structure, in particular a plurality of defined data items. According to some examples, each data item may define an image acquisition parameter, such as the settings used, the body part examined, the image acquisition protocol, the diagnostic background and the like. According to some examples, the scan geometry data, in particular the data items of the scan geometry data, may comprises an angle, a slice information, a slice alignment or a field of view setting. According to some examples, the scan geometry data comprises one or more image acquisition parameters with which or based on which the medical image, in particular the subsequent scan data, can been acquired, such as settings of the medical imaging system and/or control data and/or signals for the imaging system. According to some examples, the scan geometry data, may be a transformation information, in particular in form of a matrix, to specify how based on the first scan data the scan geometry of a subsequent scan needs to be adjusted. In particular, this transformation information comprised in the scan geometry data may be a transformation matrix.

In general, a trained machine learning model mimics cognitive functions that humans associate with other human minds. In particular, by training based on training data, the trained machine learning model is able to adapt to new circumstances and to detect and extrapolate patterns. Other terms for a trained machine learning model may be machine-learned function, trained function, trained mapping specification, mapping specification with trained parameters, function with trained parameters, algorithm based on artificial intelligence, or machine learned algorithm.

In general, parameters of a trained machine learning model can be adapted via training. In particular, supervised training, semi-supervised training, unsupervised training, reinforcement learning and/or active learning can be used. Furthermore, representation learning (an alternative term is “feature learning”) can be used. In particular, the parameters of the machine-learned function can be adapted iteratively during training.

In particular, a trained machine learning model can comprise a neural network, a support vector machine, a decision tree and/or a Bayesian network, and/or the trained machine learning model can be based on k-means clustering, Q-learning, genetic algorithms and/or association rules. In particular, a neural network can be a deep neural network, a convolutional neural network or a convolutional deep neural network. Furthermore, a neural network can be an adversarial network, a deep adversarial network and/or a generative adversarial network.

The trained machine learning model may be generally configured to determine scan geometry data based on information obtained from the first scan data. For instance, the trained machine learning model may be configured to extract one or more features from an image and/or text information contained in the first scan data, for example a DICOM file, and determine scan geometry data for obtaining subsequent scan data using the imaging system.

Thus, the trained function may comprise a feature extractor and a classifier. In particular, the feature extractor and the classifier may be implemented as a neural network, in particular, a convolutional neural network, with some network layers trained to extract features and other network layers being trained to provide a classification according to the most likely scan geometry data. Further, the scan geometry data may be obtained by applying a natural language processing function to the data, in particular non-image data, contained within the first scan data. According to some examples, the natural language processing function may be based on a large language model.

According to some examples, however, the trained machine learning model comprises at least one of: a convolutional neural network, a transformer network, in particular, a vision transformer, and/or a FocalNet.

A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks, input image data, in particular first scan data, and/or additional other data can be processed in a very efficient way, because a convolution operation based on different kernels can extract various features from the image data. By adapting the weights of the convolution kernel, the relevant features can be readily found during training. Furthermore, based on the weight-sharing in the convolutional kernels, less parameters need to be trained, which prevents overfitting in the training phase and allows to have faster training or more layers in the network, improving the performance of the network.

A transformer network is a neural network architecture generally comprising an encoder, a decoder, or both an encoder and decoder.

In some instances, the encoders and/or decoders are composed of several corresponding encoding layers and decoding layers, respectively. Within each encoding and decoding layer is an attention mechanism. The attention mechanism, sometimes called self-attention, relates data elements (such as words or pixels) within a series of data elements to other data elements within the series.

The encoder, in particular, may be configured to transform the input image data, in particular first scan data, into a numerical representation. The numerical representation may comprise a vector per input token (e.g., per image patch). The encoder may be configured to implement an attention mechanism so that each patch is affected by the other patches in the input. In particular, the encoder may be configured such that the representations resolve the desired output, i.e., the scan geometry data of the trained function.

The decoder, in particular, may be configured to transform an input into a sequence of output tokens. In particular, the decoder may be configured to implement a masked self-attention mechanism so that each vector of a token is affected only by the other tokens to one side of a sequence. Further, the decoder may be auto-regressive meaning in that intermediate results (such as a previously predicted sequence of tokens) are fed back.

According to some examples, the output of the encoder is input into the decoder.

Further, the transformer network may comprise a classification module or unit configured to map the output of the encoder or decoder to a set of learned outputs in the form of the scan geometry data.

In particular, the transformer network may be embodied as a vision transformer. The vision transformer may be configured to break down input image data, in particular first scan data into patches and tokenize them (extracting representation vectors), before applying the tokens to a standard transformer architecture. The vision transformer may comprise an attention mechanism configured to repeatedly transform representation vectors of image patches for incorporating more and more semantic relations between image patches in an image.

According to some examples, the vision transformer may be obtained by training a masked autoencoder. A masked auto-encoder comprises two vision transformers put end-to-end. The first one takes in image patches with positional encoding, and outputs vectors representing each patch. The second one takes in vectors with positional encodings and outputs image patches again. During training, both vision transformers are used. An image is cut into patches. The second vision transformer takes the encoded vectors and outputs a reconstruction of the full image. During use, the first vision transformer may be used as encoder and/or the second vision transformer may be used as generative AI function.

For a review on transformer networks, reference is made to Vaswani et al., “Attention Is All You Need”, in arXiv: 1706.03762, Jun. 12, 2017, the contents of which are herein included by reference in their entirety.

An advantage of transformer networks is that, due to the attention mechanism, transformer networks can efficiently deal with long-range dependencies in input data. Further, encoders used in transformer networks are capable of processing data in parallel which saves computing resources in inference. Moreover, decoders of transformer networks, due the auto-regression, are able to iteratively generate a sequence of output tokens with great confidence.

Obtaining, from a medical imaging system, first scan data of the subject may comprise the initial acquisition of scan data, in particular first scan data, from the subject using a medical imaging system. The first scan data may provide a baseline image data of the subject, which can be used for subsequent imaging and processing. The first scan data may be obtained with first scan geometry data being different from the scan geometry determined by the trained machine learning model. To put it differently, the initial scan (or first scan) may be performed using standard image acquisition and/or slice alignment parameters, in particular to determine the region of interest.

Providing a trained machine learning model configured to determine, based on first scan data, scan geometry data for a subsequent scan may comprise providing a with extensive medical imaging data preconditioned machine learning model. This model may be specifically configured to analyze the first scan data and determine a scan geometry for a subsequent scan, in particular optimized for a post-processing task. The model may utilize one or multiple algorithms to identify key features, patterns or view settings in the first scan data. Based on these identified features, patterns and/or settings the model may be configured to suggest and/or predict parameters for subsequent imaging, in particular subsequent scans.

Applying the trained machine learning model to the first scan data to obtain scan geometry data for a subsequent scan of the subject may involve processing the initial images, in particular the first scan data, through the trained machine learning model, which uses its trained capabilities to extract relevant features and predict the suitable scan geometry for the subsequent scan. To put it differently, the parameters, in particular the imaging acquisition parameters, of the subsequent scan are determined based on the initial scan data with the model to generate subsequent scan data, in particular adapted to a post-processing task.

Obtaining, from the imaging system, subsequent scan data of the subject using the scan geometry data involves using the determined optimized parameters provided by the machine learning model to capture new scan data, in particular images. It may include inputting the scan geometry into the imaging system in order to control the imaging system so as to obtain the subsequent scan data.

Providing the subsequent scan data entails making the subsequent scan data available for further analysis or diagnosis. This involves processing and storing the new images captured during the second scan, ensuring they are accessible for medical professionals.

The outlined computer-implemented method may provide significant advantages in the context of medical imaging by incorporating a trained machine learning model to optimize scan parameters. By capturing detailed images with optimized scan geometry data sufficient and tailored image data may be gathered for downstream tasks and workflow automation within medical and/or clinical processes. The subsequent scan data may be expected to be of higher quality and more targeted, offering improved diagnostic information about the subject. Furthermore, the automation of scan geometry data determination reduces the manual workload on radiologists and technicians, streamlining the overall workflow and allowing medical professionals to focus on more critical tasks. Consequently, the method contributes to quicker turnaround times for imaging results, enhancing the efficiency of the diagnostic process.

According to one aspect, providing the trained machine learning model configured to determine the scan geometry data for a subsequent scan may further be based on imaging data. Imaging data may in particular comprise sensor data recorded during imaging, in particular the first scan and/or subsequent scan. For example, the imaging data may comprise sensor data from a Hall sensor, in particular of the local coils, and/or 3D camera data. The trained machine learning model may be applied to the imaging data and/or the first scan data to obtain scan geometry data for a subsequent scan of the subject. In other words, the input data for the trained machine learning model may also comprise other data related to the image data and/or the imaging process to determine the scan geometry data.

With this the trained machine learning model advantageously may be provide provided with additional information to determine the scan geometry data, which may be beneficial for the quality and accuracy of the subsequent scan, in particular with regard to the post-processing function.

According to one aspect, the subsequent scan data is to be processed by a post-processing function, the post-processing function being configured to generate post-processed data based on the subsequent scan data. The trained machine learning model is configured such that the scan geometry data is adapted to the post-processing function.

The trained machine learning model is configured to adapt and/or determine the scan geometry data dependent on a post processing function. The trained machine learning model is configured to output scan geometry data optimized for the post-processing function. The post processed data and/or the resulting data of the post processing function may determine and/or affect the loss function of the trained machine learning model.

According to an aspect, the method further comprises providing a post processing function and/or applying the post processing function.

Post processing of medical images and/or scan data in general refers to one or more operations, methods and/or functions applied to medical images and/or subsequent scan data to enhance, analyze, and interpret the medical image data and/or scan data, in particular for diagnosis. Preferably the post processing function is configured to generate post-processed data (also post-processing data), which may include refined images, quantitative measurements, or diagnostic insights derived from the subsequent scan. The post-processing function may require the subsequent scan data as input data. The post-processing function may require additional data, such as e.g. the scan geometry data, as input data. The post-processing function and/or method may comprise applying one or more algorithms and/or image/data processing techniques to enhance the data quality and/or extract critical diagnostic information. The post processing function may be specifically configured to process subsequent scan data optimized with the scan geometry data provided by the trained machine learning model, thereby generating optimized post-processed data. Post-processed data refers to the refined output data generated through one or more operations, techniques and/or functions applied to images and/or scan data obtained from medical imaging systems, such as MRI or CT scans, derived after the initial imaging process and/or first scan. For instance, in the context of MRI, a post-processing step may involve the application of algorithms to improve image clarity, correct distortions, or extract specific anatomical features of an MRI scan. An example for a post processing function may be a segmentation function for deriving a specific region from MRI scans. The specific regions may be for example be a tumor and/or organ. Another example involves the application of image registration techniques to align images from different scans for comparison or combination. The generation of three-dimensional reconstructions from two-dimensional slice data and/or providing a comprehensive view of the anatomical structure can be further examples of post processing processes and/or functions.

The method may ensure that subsequent scan data is robust and well-suited for post-processing functions. The high-quality, targeted nature of the subsequent scan data enables post-processing functions to generate more accurate and detailed results, which is particularly valuable for advanced imaging techniques and complex diagnostic tasks. This robustness in post-processed data may enhance the precision and reliability of medical diagnoses, ultimately improving patient outcomes.

According to one aspect, at least one parameter of the machine learning model is determined based on output data of the post-processing function.

According to one aspect, the trained machine learning model has been trained using a loss function based on the post-processed data.

The machine learning model's parameters may be intricately tied or linked to the output data of the post-processing function through a feedback loop. Specifically, the machine learning model may utilize a loss function that integrates and/or is calculated with the post-processed data. For instance, during the training phase, the model may be exposed to a series of scan data, which it processes to generate optimized scan geometry data. This data, applied in a subsequent scan, may yield subsequent scan data that undergoes post-processing. The refined output, in particular post-processing data may feed back into the model's training method and/or cycle. Here, a gradient-based backpropagation may be employed, whereby the differences between the post-processing data and the ground truth may guide the adjustments in the model parameters.

At least one parameter of the machine learning model is determined based on the output data of the post-processing function by incorporating a feedback loop. This may lead to quality and accuracy of the post-processed data influencing the training process of the model. Specifically, once the post-processing function generates refined images or diagnostic insights, these outputs are analyzed to assess the effectiveness and/or quality of the scan geometry data. Metrics derived from the post-processed data, such as image clarity, segmentation accuracy, and diagnostic reliability, may be used to adjust the parameters of the machine learning model. A gradient-based backpropagation may ensure that the model learns to output scan geometry data that is optimized for the specific requirements of the post-processing function. Through iterative training, the parameters of the machine learning model may be iteratively adapted and/or fine-tuned.

By optimizing the machine learning model parameters based on the post-processed data, the method may ensure that the scan geometry is consistently aligned with the needs of advanced imaging techniques and or systems, leading to higher quality and more detailed images. This may enhance the overall quality of the subsequent scan data, ensuring that it is well-suited for (high precision) post-processing tasks. The training of the machine learning model based on post-processed data reduces the variability in image quality and enhances the reliability of diagnostic information derived from the scans. Furthermore, the integration of post-processing feedback into the model training process streamlines the imaging workflow, reducing the need for manual adjustments and enabling radiologists and technicians to focus on critical interpretive tasks.

According to one aspect, the first scan data comprise image data from an initial scan. The subsequent scan data comprise image data from a subsequent scan. The initial scan is obtained based on initial scan parameters. The subsequent scan is obtained based on subsequent scan parameters. The subsequent scan parameters are determined based on the scan geometry data.

According to one aspect, the initial scan to obtain the first scan data and the subsequent scan to obtain the subsequent scan data differ in at least one of resolution parameters, imaging area and/or region of interest, and/or scan geometry.

The initial scan may in particular refer to the first set of image data acquired during a medical imaging procedure. This initial scan may serve as the baseline or reference data for subsequent imaging/scans. The initial scan parameters may be predefined. The initial scan parameters may be obtained by inputting and/or analyzing of patient data, such as weight, height, medical condition and/or diagnosis for example. Initial scan parameters may comprise specific settings and/or conditions used to perform the initial scan, for example a MRI localizer scan. These parameters may include technical specifications such as the type of imaging modality, slice thickness, resolution, contrast settings, and any other relevant factors that influence the quality and characteristics of the initial imaging data.

The subsequent scan may in particular be an additional set of image data acquired after the initial scan. The subsequent scan may also be characterized as diagnostic scan. This scan may be performed for example to capture further details of a region of interest, monitor changes of a finding and/or ROI, or provide detailed images based on findings, ROI and/or settings from the initial scan. Subsequent scan parameters may be settings and/or conditions used to perform the subsequent scan, for example a MRI scan. Subsequent scan parameters may be determined based on scan geometry data and/or data and/or settings from the initial scan. Subsequent scan parameters may be adjusted automatically and/or manually, in particular to optimize the imaging process. These adjustments may include changes in slice spacing, resolution, imaging angles, and other technical aspects to enhance the accuracy and detail of the subsequent imaging data.

Based on the scan geometry data a three-dimensional volume and/or region of interest (ROI) of the subject may be defined. The initial scan data may be also referred to as localizer scan data. The scan geometry data may also be denoted as scan geometry, slice geometry (data) and/or scan coordinates. The initial scan for example might have a lower resolution setting and/or a bigger image space compared to the subsequent scan.

By tailoring subsequent scan parameters based on initial scan geometry, the imaging process may become more adaptive and targeted, allowing for better visualization of critical areas (in particular ROI). This method may not only improve diagnostic accuracy but may also optimize the overall efficiency of the imaging procedure, reducing the need for repeat scans and minimizing patient exposure to unnecessary imaging.

According to one aspect, the scan geometry data comprise an align-matrix. The first scan data comprises initial scan parameters. The align-matrix comprises alignment information for obtaining the subsequent scan data with respect to the initial scan parameters.

The align-matrix, also referred to as Auto Align Matrix (AAM) or matrix, in particular may be a rigid transformation matrix. The align-matrix may be used for slice alignment of a medical imaging system, in particular an MRI system. The align-matrix may be structured to transform the slice direction from the first scan to the subsequent scan to achieve the planned alignment. The align-matrix may comprise, in particular consist of, rotation and translation components. The rotation and translation components for example may represent how the spatial coordinates of the first scan need to be adjusted to match the desired alignment of the diagnostic scan. This matrix may consist of rows and columns, for example a 4×4 matrix. The align-matrix may be calculated and/or obtained with the trained machine learning model. The trained machine learning model may predict the align-matrix by learning from training data, including pairs of initial scans and corresponding scan geometry data and/or subsequent scan data, for example optimal diagnostic scan alignments. During training, the trained machine learning model may be updated iteratively by backpropagating a segmentation quality metric. This quality metric may provide a gradient that guides the network in minimizing the target loss, thereby refining the predicted alignment matrix.

The align-matrix may be applied in particular in MRI slice planning to ensure that the slices obtained during the diagnostic scan are optimally aligned for subsequent segmentation tasks. By applying the align-matrix, clinicians can transform the initial scan directions to match the planned diagnostic scan alignment, enhancing the accuracy and quality of the imaging results. This precise alignment is crucial for accurate diagnosis and effective clinical decision-making.

According to one aspect, the method further comprises reporting the scan geometry data to a user via a user interface.

The user interface may for example be an integrated display system within the medical imaging device. The user interface may serve as an interactive platform for healthcare professionals to view and manipulate the scan geometry data. The user interface can in particular be designed to display a align-matrix and provide options to the user to adjust the align-matrix, scan geometry data and/or scan parameters of the subsequent scan based on the first scan data and/or the predicted scan geometry data. The user interface can also allow a user to confirm, in particular via the trained machine learning model, predicted scan geometry data. Furthermore, the user interface can also allow a user to reobtain or to initiate another predicting step of the scan geometry data. The user interface may include graphical elements like buttons, sliders, and visual feedback mechanisms that allow for easy interaction and adjustment of scan geometry data and/or scan settings.

For example, during an MRI imaging, the initial scan data is processed to generate scan geometry data in form of an align-matrix. The align-matrix can be presented to a user via the user interface. This may allow the user to visualize the alignment based on the scan geometry data and to adjust the subsequent scan parameters, such as modifying the slice thickness or changing the imaging angle, before initiating the subsequent scan.

By informing the user of the scan geometry data via the user interface the user might be enabled to conduct tasks during the medical imaging workflow in a more efficient matter.

According to one aspect, the method further comprises receiving a user input to provide additional scan geometry data via a user interface. Obtaining the subsequent scan data, from a medical imaging system, is additional based on the additional scan geometry data.

One step may be directed to providing a user information based on the scan geometry data to a user via a user interface. One step may be directed to obtain a user input related to the user information from the user via the user interface. One step is directed to determine the additional scan geometry data based on the user input.

The user information may comprise a numerical value or a graphical indicator reflecting the scan geometry data. The user information may provide feedback to the user about the reliability and/or accuracy of the scan geometry data, and whether it is sufficient for further processing (e.g. imaging). The user information may also comprise possible actions for the user to take, such as confirming, correcting, supplementing the scan geometry data, or requesting additional information from a database or another source. The user information may also provide the user with a choice between multiple scan geometry datasets as the right input for subsequent scan, in particular in case of a discrepancy or uncertainty. The user information may therefore, for example, comprise confidence scores and/or information which of the scan geometry data is deemed to be optimal for the subsequent scan. The user information may be displayed in the same interface as a representation of the medical image, or in a separate interface. The user information may be updated dynamically as the scan geometry data changes or is verified. The user input may be received through various interface devices and/or interface means, such as keyboard, mouse, touch screen, voice command, or gesture recognition. The user input may update a confidence score, scan settings and/or the scan geometry data. In particular, the user input may be directed to confirming, correcting, supplementing the scan geometry data, requesting additional information from the database or another source and/or making a selection between for example a first and second scan geometry dataset.

By enabling the user to conduct (real-time) adjustments based on reported scan geometry and/or by presenting adjustment options to the user, the need for repeated scans and/or the change of an erroneous scan, for example with wrong slice alignment may be reduced. This may increase efficiency and also translate to lower overall costs for the medical imaging and diagnosis process and better allocation of imaging resources, ultimately improving patient care and operational efficiency.

According to one aspect, a computer-implemented method for providing a trained machine learning model is provided. The input training data comprising first scan training data, subsequent scan training data, post-processing training data is received. The scan geometry data is predicted by applying the trained machine learning model to the first scan training data. Aligned subsequent scan data is obtained by applying the scan geometry data to the subsequent scan training data to simulate a scan. Post-processing data is obtained by inputting the aligned subsequent scan data into a post-processing function, the post processing function being configured to generate post-processed data based on the aligned subsequent scan data. The trained machine learning model is adapted based on a comparison of the post processing data and the post processing training data. The trained machine learning model is provided.

The input training data may further comprise scan geometry training data and/or a post-processing quality training metric. The input data may be received from a database or any other data storage medium. The predicted scan geometry data may be applied to the subsequent scan training data. In other words, the subsequent scan training data may be viewed in a direction of the predicted scan geometry data. The subsequent scan training data may be sliced and/or interpolated with the scan geometry data. The slicing may be based on the potentially anisotropic target volume resolution. The slicing and/or interpolation may be implemented differentiable. A differential slicing and/or interpolation can allow a backpropagating of a gradient for every step. The trained machine learning model may comprise a deep learning framework to implement the backpropagation.

A post-processing quality metric may be further determined by comparing the post-processing data to the post-processing trainings data. The post-processing quality metric may be minimized by backpropagating the previous steps to optimize one or more parameters of the trained machine learning model.

A post-processing quality metric may be predicted between the output of the post processing function and the ground truth, in particular the post-processing training data, during the training phase. The metric may represent the loss scalar that is minimized during the training to optimize the trained machine learning model.

The quality metric (also loss metric) may be backpropagated through all previous processing steps into the trained machine learning model. The parameters (or weights) of the trained machine learning model may be updated in negative gradient direction using an optimizer. The optimizer may be for example a stochastic gradient descent or an Adam optimizer.

Training the trained machine learning model in regard to post processing data and/or a quality metric of a post processing function allows for a refined data input for the post processing function. Ultimately, this may lead to quality and task-adapted post-processed data, essential for medical diagnosis.

According to one aspect, obtaining the aligned subsequent scan data comprises planning a scan direction in an isotropic volume of the subsequent scan training data based on the scan geometry data and/or simulating the subsequent scan based on the scan geometry data and/or the scan direction to receive an anisotropic volume slicing.

The subsequent scan training data may be sliced and/or interpolated with the scan geometry data. The slicing may be based on the potentially anisotropic target volume resolution. The slicing and/or interpolation may be implemented differentiable. A differential slicing and/or interpolation can allow a backpropagating of a gradient for every step. The slicing may simulate the clinical image acquisition, in particular the subsequent scan, in direction of the scan geometry data during the training of the trained machine learning model.

The described aspect may ensure precise slice alignment and accurate representation of a target imaging volume, resulting in reliable and consistent subsequent scans. Additionally, the training process of the machine learning model may be enhanced by effectively simulating the clinical image acquisition, leading to an improved performance of the trained machine learning model.

According to one aspect, the post-processing function comprises one or more parameters. The one or more parameters of the post-processing function are adapted during the training of the trained machine learning model.

A pre-trained deep neural network may be applied to the scan geometry data direction sliced data, in particular MRI volume, as a post-processing function to obtain post processing data, e.g. a segmentation mask of an anatomy of interest.

According to one aspect the parameters of the post-processing function may be constant, unchangeable, non-editable and/or frozen during the training process. However, one or more parameters of the post-processing function may be adapted during and/or after the training of the trained machine learning model. The training may for example comprise several training phases. In these different training phases, the parameters of the trained machine learning model and post-processing function may be adapted in an alternating way. In a first training phase for example the post-processing function parameters may be kept constant and/or non-editable. In a second training phase for example the parameters of the trained machine learning model may be kept constant and/or non-editable. In other words, as one function or model is kept constant the other is optimized during the training.

By dynamically adjusting parameters of the post-processing function during the training of the trained machine learning model the post-processing functions can be improved and adapted to the input data, in particular the subsequent scan data based on the scan geometry data. This may allow for an iterative optimization process ensuring that the model is aligned with the input data distributions and complexities, ultimately leading to improved performance of the trained machine learning model itself.

According to one aspect, the trained machine learning model for the computer-implemented method for providing subsequent scan data of a subject according to any one of the previous aspects is provided the computer-implemented method for providing a trained machine learning model according to any one of the previous aspects.

By applying the provided trained machine learning model the method allows for an efficient and reliable provision of subsequent scan data and/or scan geometry data.

According to one aspect, a medical imaging system is provided. The medical imaging system comprises a scan unit, a computing unit and a post-processing unit. The scan unit is configured to acquire first scan data of a subject. The computing unit is configured to obtain scan geometry data from the first scan data with a trained machine learning model. The scan unit is further configured to acquire a subsequent scan data of the subject based on the scan geometry data. The post-processing unit is configured to output post-processed data from the subsequent scan data with a post-processing function. The trained machine learning model is configured to output scan geometry data tailored for the post-processing function.

The scan unit may comprise a medical imaging modality to generate or produce medical image data. For example, a medical imaging modality may be a computed tomography system (CT system), a magnetic resonance system (MR system), an angiography (or C-arm X-ray) system, a positron-emission tomography system (PET system) or the like. Computed tomography is a widely used imaging method making use of “hard” X-rays produced and detected by a spatially rotating instrument. Magnetic resonance imaging (MRI) is a typically a non-invasive medical imaging technology utilizing magnetic fields and radio waves to picture the response of molecules in a patient's body. Attenuation data (also referred to as raw data) derived from these modalities may be processed by a computed analytic software producing detailed images of the internal structure of the patient's body parts. The produced sets of images may be called scans and may constitute multiple series of sequential images to present the internal anatomical structures in cross sections perpendicular to the axis of the human body. The scan units may comprise detectors and/or antennas to receive attenuation data and/or signals.

The computing unit may be realized as a data processing system or as a part of a data processing system. Such a data processing system can, for example, comprise a cloud-computing system, a computer network, a computer, a tablet computer, a smartphone and/or the like. The computing unit can comprise hardware and/or software. The hardware can comprise, for example, one or more processors, one or more memories and combinations thereof. The one or more memories may store instructions for carrying out the method steps according to one or more example embodiments of the present invention. The hardware can be configurable by the software and/or be operable by the software. Generally, all units, sub-units, or modules may at least temporarily be in data exchange with each other, e.g., via a network connection or respective interfaces. Consequently, individual units may be located apart from each other.

The post-processing unit may be realized as part of a data processing system, similar to the computing unit. It may comprise components, like a processor for example, to execute functions, in particular trained functions, designed to process subsequent scan data to generate post-processed data, such as segmentation masks or enhanced images for example. The post processing unit may comprise one or more processors and/or memory components configured to store functions, algorithms and/or instructions critical for post-processing methods and/or operations. The post processing unit might be configured to comprise and run a pre-trained deep neural network to adapt, annotate and/or refine the subsequent scan data acquired by the scan unit. The post-processing unit may comprise advanced image processing algorithms and/or functions tailored for specific medical imaging modalities and/or tasks. The post-processing unit may operate independently or in conjunction with the computing unit. The post-processing unit may communicate with other units of the medical imaging system via network connections or interfaces allowing for data exchange and/or transmission.

According to other aspects, the system is adapted to implement the inventive method in their various aspects for providing subsequent scan data of a subject. The advantages described in connection with the method aspects may also be realized by the correspondingly configured systems' components.

According to another aspect, the present invention is directed to a computer program product comprising program elements which induce a computing unit of systems herein described to perform the steps according to one or more of the above method aspects and examples as herein described, when the program elements are loaded into a memory of the computing unit.

According to another aspect, the present invention is directed to a computer-readable medium on which program elements are stored that are readable and executable by a computing unit of systems herein described to perform the steps according to one or more method aspects and examples as herein described, when the program elements are executed by the computing unit.

The realization of one or more example embodiments of the present invention by a non-transitory computer program product and/or a non-transitory computer-readable medium has the advantage that already existing providing systems can be easily adapted by software updates in order to work as proposed by one or more example embodiments of the present invention.

The computer program product can be, for example, a computer program or comprise another element next to the computer program as such. This other element can be hardware, e.g., a memory device, on which the computer program is stored, a hardware key for using the computer program and the like, and/or software, e.g., a documentation or a software key for using the computer program. The computer program product may further comprise development material, a runtime system and/or databases or libraries. The computer program product may be distributed among several computer instances.

### BRIEF DESCRIPTION OF THE DRAWINGS

Characteristics, features and advantages of the above-described invention, as well as the manner they are achieved, become clearer and more understandable in the light of the following description of embodiments, which will be described in detail with respect to the figures. This following description does not limit the present invention on the contained embodiments. Same components, parts or steps can be labeled with the same reference signs in different figures. In general, the figures are not drawn to scale. In the following:

FIG. 1 schematically depicts a computer-implemented method for providing subsequent scan data of a subject according to an embodiment,

FIG. 2 schematically depicts data flows in a computer-implemented method for providing subsequent scan data of a subject according to an embodiment,

FIG. 3 schematically depicts a computer-implemented method for providing a trained machine learning model according to an embodiment,

FIG. 4 schematically depicts data flows in a computer-implemented method for providing a trained machine learning model according to an embodiment,

FIG. 5 schematically depicts a trained machine learning model for determining scan geometry data,

FIG. 6 schematically depicts a medical imaging system.

### DETAILED DESCRIPTION

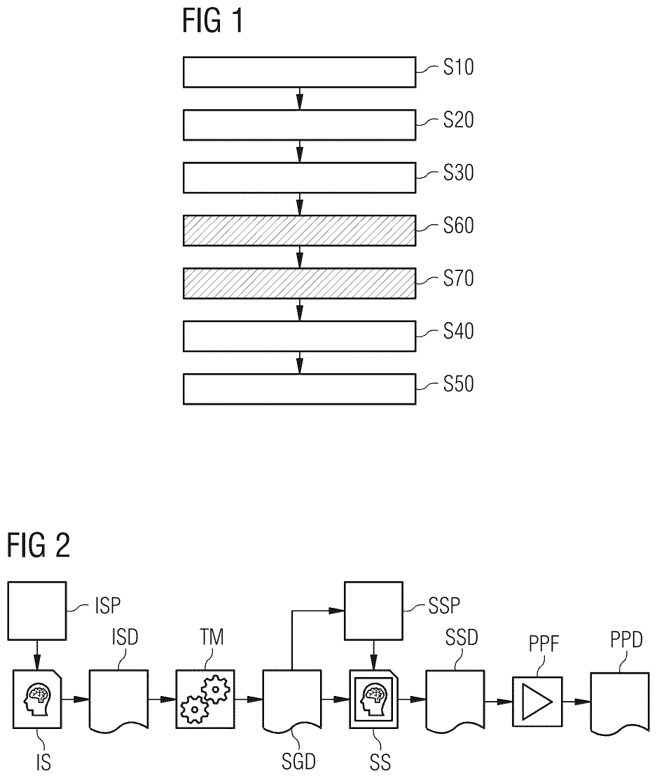

FIG. 1 depicts a computer-implemented method for providing subsequent scan data of a subject according to an embodiment. Corresponding data streams are illustrated in FIG. 2. The method comprises several steps. The order of the steps does not necessarily correspond to the numbering of the steps but may also vary between different embodiments of the present invention. Further, individual steps or a sequence of steps may be repeated.

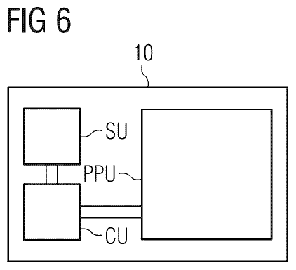

At S10, first scan data IS of a subject is obtained. The first scan data IS may comprise image data. The first scan data ISD may also be referred to as initial scan data or localizer data. Obtaining the first scan data ISD may involve selecting image data from a plurality of image data sets, e.g., stored in the database. The selection may be performed manually by the user, e.g., by selecting appropriate image data in a graphical user interface running in a user interface. Alternatively, the first scan data ISD may be provided to a computing unit CU (see FIG. 6) by the user by way of uploading the first scan data ISD to the computing unit CU. Alternatively, the first scan data ISD may be provided to the computing unit CU by a scan unit SU via a data connection. According to an example, the first scan data ISD has been acquired using a magnetic resonance imaging modality using initial scan parameters ISP, for example a particular image weighting and/or magnetic resonance sequence. The image data may be temporarily stored in a digital format at the computing unit CU, e.g., in a working memory of the computing unit CU. The image data of the first scan data ISD may be extracted. According to other examples, a representative part of the image data of the first scan data may be selected, in particular an image slice or a region of interest, and only the representative part may be extracted.

At S20, a trained machine learning model TM is provided that is configured to determine to determine scan geometry data SGD for a subsequent scan SS based on first scan data IS. The trained machine learning model TM may be a machine learning model, such as a neural network, a convolutional neural network or any other suitable algorithm, which has been trained on a set of first scan data ISD and verified corresponding scan geometry data SGM and/or subsequent scan data SSD. The trained machine learning model TM may be hosted at the computing unit CU or at a remote server that is accessible via a network connection. The trained machine learning model TM may receive first scan data ISD, in particular image data of the first scan data IS, as an input and output scan geometry data SGD as an output.

At S30, a scan geometry data SGD is determined by applying the trained machine learning model TM to the first scan data ISD. The trained machine learning model TM may extract relevant features from the first scan data ISD that are indicative of the scan geometry and/or slice alignment, such as edges, contours, textures, colors, shapes, regions, or any other visual elements. The relevant features may be extracted using image processing techniques, such as filtering, segmentation, feature detection, feature extraction, or any other suitable methods. The relevant features may be encoded in a vector or a matrix format that can be input to the trained machine learning model TM. The trained machine learning model TM may output a probability distribution over a set of possible and/or suitable scan geometry data, with features and/or categories such as image direction, image contrast, image resolution, image orientation, image slice, image region of interest, or any other suitable scan features. The trained machine learning model TM may select the most probable information for each category and/or each relevant feature and/or datapoint of the scan geometry data. The scan geometry data SGD may comprise the selected scan geometry data categories and their corresponding values or labels. The scan geometry data SGD may be stored in a digital format at the computing unit CU.

At S40, subsequent scan data SSD of a subject is obtained. The subsequent scan data SSD may comprise image data. The subsequent scan data SSD may also be referred to as diagnostic scan and/or medical image data. Obtaining the subsequent scan data SSD may involve selecting image data from a plurality of image data sets, e.g., stored in the database. The selection may be performed manually by the user, e.g., by selecting appropriate image data in a graphical user interface running in a user interface. Alternatively, the subsequent scan data SSD may be provided to a computing unit CU (see FIG. 6) by the user by way of uploading the subsequent scan data SSD to the computing unit CU. Alternatively, the subsequent scan data SSD may be provided to the computing unit CU by a scan unit SU via a data connection. According to an example, the subsequent scan data SSD has been acquired using a magnetic resonance imaging modality using subsequent scan parameters SSP and/or the scan geometry data SGD. The subsequent scan parameters SSP may be adapted based on the scan geometry data SGD. The subsequent scan data SSD may be temporarily stored in a digital format at the computing unit CU, e.g., in a working memory of the computing unit CU.

At S50, the subsequent scan data SSD is provided. This may involve showing the subsequent scan data SSD and/or the scan geometry data SGD in a user interface, e.g., in a suitable graphical user interface. Moreover, S50 may comprise providing the subsequent scan data SSD for subsequent image processing steps, in particular post processing steps, as shown in connection with FIG. 2.

Steps S60 and S70 may be optional steps. Preferably those steps S60, S70 are executed before step S40. However, it is also conceivable that the steps are executed in a different order than depicted in FIG. 1.

At S60 an information, in particular the scan geometry data SGD, may be provided to a user via an interface. The information may indicate to the user whether the scan geometry data SGD is suitable and/or correct for a subsequent scan or not, and to what extent. The information may also suggest or recommend a course of action for the user, such as accepting, rejecting, modifying, or confirming the scan geometry data SGD. The information may be displayed in the interface in a suitable format, such as image, text, symbols, colors, graphs, or any other visual elements.

At S70, a user input related to the information, in particular the scan geometry data SGD, may be obtained from the user via the interface. The user input may express the user's decision or preference regarding the scan geometry data SGD, such as whether the user agrees or disagrees with the information, or whether the user wants to change, update, or confirm the information. The user input may also provide feedback or corrections to the scan geometry data SGD, such as adding, deleting, or modifying the scan geometry datapoints, categories or values. The user input may be received by the interface in a suitable format, such as text, symbols, colors, gestures, voice commands, or any other inputs. Based on the user input, the scan geometry data SGD may be determined or adjusted accordingly.

According to some examples a post-processing step might be comprised. The post-processing step may comprise inputting the subsequent scan data into a post-processing function PPF. The post-processing function PFF may output post-processing data PPD based on the subsequent scan data. The post-processing data PPD may be temporarily stored in a digital format at the computing unit CU, e.g., in a working memory of the computing unit CU.

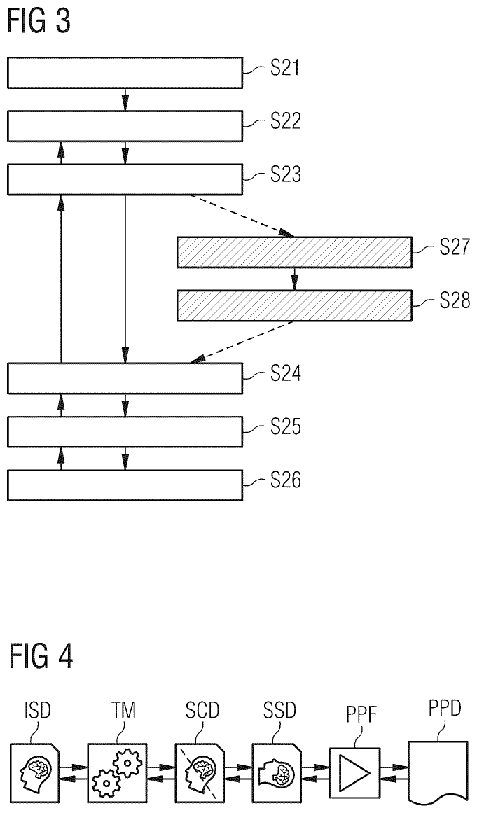

FIG. 3 depicts a computer-implemented method for providing a trained machine learning model according to an embodiment. Corresponding data streams are illustrated in FIG. 3. The method comprises several steps. The order of the steps does not necessarily correspond to the numbering of the steps but may also vary between different embodiments of the present invention. Further, individual steps or a sequence of steps may be repeated.

At S21, input training data is received. The input training data may comprise first scan training data, subsequent scan training data and/or post-processing training data. Receiving the input training data first scan data may be performed manually by the user, e.g., by uploading the input training data or downloading input training data from a data storage. The input training data may be provided to a computing unit CU hosting the (pre-trained) machine learning model via a data connection. The input training data may be temporarily stored in a digital format at the computing unit CU, e.g., in a working memory of the computing unit CU.

At S22, scan geometry data is predicted by applying the trained machine learning model to the first scan training data. The prediction of the scan geometry data may involve processing the first scan training data through a series of computational layers within the machine learning model. The trained machine learning model may be configured to analyze the input data using one or more functions to derive the scan geometry data. The scan geometry data may comprise the spatial configuration and/or parameters required for the subsequent scan. The predicted scan geometry data may be temporarily stored in the computing unit CU's memory for the following steps. The scan geometry data may comprise the precise slice direction of the subsequent scan. For example, a rigid transformation matrix, denoted as align-matrix, is predicted as scan geometry data. The align-matrix may define how the first scan slice direction must be transformed to receive the planned alignment of the subsequent scan.

At S23, aligned subsequent scan data is obtained by applying the scan geometry data to the subsequent scan training data to simulate a scan.

Step S23 may comprise steps S27 and/or S28. Steps S27 and/or S28 are optional steps. Preferably those steps S27, S28 are executed before step S24.

Step S23 may involve the simulation of the subsequent scans using the predicted scan geometry to align the slices of the subsequent scan data. This may involve serval subsets for example the planning of a scan direction. The subsequent scan training data may comprise scan data corresponding to the first scan training data. However, the subsequent scan training data may have been acquired using standard scan parameters to obtain an isotropic volume.

At S27, a scan direction SCD in an isotropic volume of the subsequent scan training data is planned based on the scan geometry data. This may allow the simulation of a subsequent scan with adapted parameters based on the scan geometry data. This step may comprise determining the optimal scan path and orientation to maximize the accuracy and efficiency of the subsequent scan. The step may be automatically executed on the computing unit CU. The scan geometry data may be analyzed by the computing unit CU and the scan direction planned accordingly.

At S28, the subsequent scan is simulated based on the scan geometry data and/or the scan direction to receive an anisotropic volume slicing. The anisotropic volume slicing may represent a subsequent scan data based on the scan geometry data. This step may comprise executing the simulation of the subsequent scan on the computing unit CU to create a scan volume with anisotropic slices, the simulated subsequent scan data SSD*.

At S24, post-processing data is obtained by inputting the aligned subsequent scan data into a post-processing function. The post processing function being configured to generate post-processed data based on the aligned subsequent scan data. The post-processing function may comprise a pre-trained deep neural network configured to predict post-processing data based on the scan direction sliced volume and/or simulated subsequent scan data SSD*. For example, may a segmentation post-processing function predict a segmentation mask of the anatomy of interest. The network parameters of the post-processing function may be frozen and/or not changed. A segmentation quality metric may be predicted between the output of the segmentation post-processing function and the ground truth segmentation mask derived from the post-processing training data. The metric represents the loss scalar that is minimized during the pipeline training to optimize the AAM prediction network.

At S25, the trained machine learning model is adapted based on a comparison of the post processing data and the post processing training data. This training and/or adaptation process may involve using a feedback mechanism known as backpropagation. In backpropagation, the difference between the predicted output (post-processing data) and the true output (post-processing training data) is calculated using a loss function. This error may then be propagated backward through the network layers to adjust the weights and biases of the trained machine learning model. The adjustments may be made in such a way that the error is minimized in future predictions. Specifically, the gradients of the loss function with respect to each weight may be computed, and the weights updated using these gradients through an optimization algorithm, such as e.g. stochastic gradient descent. By iteratively performing this process, the model's parameters are fine-tuned, thereby improving its accuracy and performance in predicting scan geometry and processing scan data.

At S26, the trained machine learning model is provided. The trained machine learning model may be stored at the computing unit CU, e.g., in a working memory of the computing unit CU and/or in a database.

In FIG. 5 an embodiment of the trained machine learning model TM is displayed. In the example shown in FIG. 5, the trained machine learning model TM is a convolutional neural network, in particular, a deep convolutional neural network. The trained machine learning model TM according is a machine learning model that can manage different types of input data. Especially image data, in particular first scan data, may be the input data of the trained machine learning model TM. The trained machine learning model TM can, in particular, process pixel or voxel values of the of the first scan data, in particular the image data of the first scan data. Of note, this is just meant as an illustrative example as the trained machine learning model TM may also be embodied by any other suitable machine learned model such as transformer architectures as elsewhere herein described.

The trained machine learning model TM according to FIG. 5 comprises convolutional layers, pooling layers and fully connected layers. In the input layer L.1, there is one node for data element, e.g. each pixel of the image data, each pixel having one channel (the respective intensity value). After the input layer, there are four convolutional layers L.2, L.4, L.6, L.8, each of the four convolutional layers is followed by a pooling layer L.3, L.5, L.7, L.9. For each of the convolutional layers, a 5×5 kernel is used (indicated by “K: 5×5”) with a padding of 2 (indicated by “P: 2”) and either one or two filters/convolutional kernels (indicated by “F: 1” or “F: 2”).

From the pooling layers L.3, L.5, L.7, L.9, the first three layers L.3, L.5, L.7 implement an averaging operation over patches of size 4×4, and the last pooling layer L.9 implements a maximum operation over patches of size 2×2. The additional layer L.10 of FIG. 6 flattens the input images data. However, this layer is not relevant for the actual calculation.

The last layers of the network are three fully connected layers L.11, L.12, L.13, the first fully connected layer having 128 input and 40 output nodes, the second fully connected layer L.12 having 40 input and 10 output nodes, and the third fully connected layer L.13 having 10 input and 2 output nodes, wherein the 2 output nodes form the output layer of the whole machine learning model.

The value of the first node of the output layer may correspond to one element of the scan geometry data (e.g. slice direction) based on the image data related to the input first scan data. The second node may relate to another element of the scan geometry data (e.g., slice thickness) and so forth. There may be as many output nodes as elements in the scan geometry data the trained machine learning model TM has to discriminate.

For training the trained machine learning model TM, a database, for example, of 500 medical images, in particular first scan training data, with confirmed scan geometry data has been used. The database was split into training data (320 datasets), validation data (80 datasets) and test data (100 datasets). That followed, the image data was extracted from the medical images, in particular first scan training data. For determining the scan geometry data, the confirmed subsequent scan training data are used. For training the trained machine learning model TM, the backpropagation algorithm was used based on a cost function L(x, y1, y2, . . . yn)=|M(x)1−y1|2+|M(x)2−y2|2+ . . . +|M(x)n−yn|2 wherein x denotes an input image data, y1 denotes whether a first element of the scan geometry data is indicated, y2 denotes whether a second element of the scan geometry data is indicated, and yn denotes whether an n-th element of the scan geometry data is indicated. Furthermore, M(x) denotes the result of applying the trained machine learning model TM to the input image data, and M(x)1, M(x)2, . . . , M(x)n correspond to the value of the first, second, . . . n-th output node if applying the trained machine learning model TM to the input image data.

Based on the validation set of 80 datasets and the corresponding annotations, the best performing trained machine learning model TM out of several machine learning models (with different hyperparameters, e.g., number of layers, size and number of kernels, padding etc.) was selected. The specificity and the sensitivity were determined based on the test set comprising 100 datasets and scan geometry data.

Wherever meaningful, individual embodiments or their individual aspects and features can be combined or exchanged with one another without limiting or widening the scope of the present invention. Advantages which are described with respect to one embodiment of the present invention are, wherever applicable, also advantageous to other embodiments of the present invention. Independent of the grammatical term usage, individuals with male, female or other gender identities are included within the term.

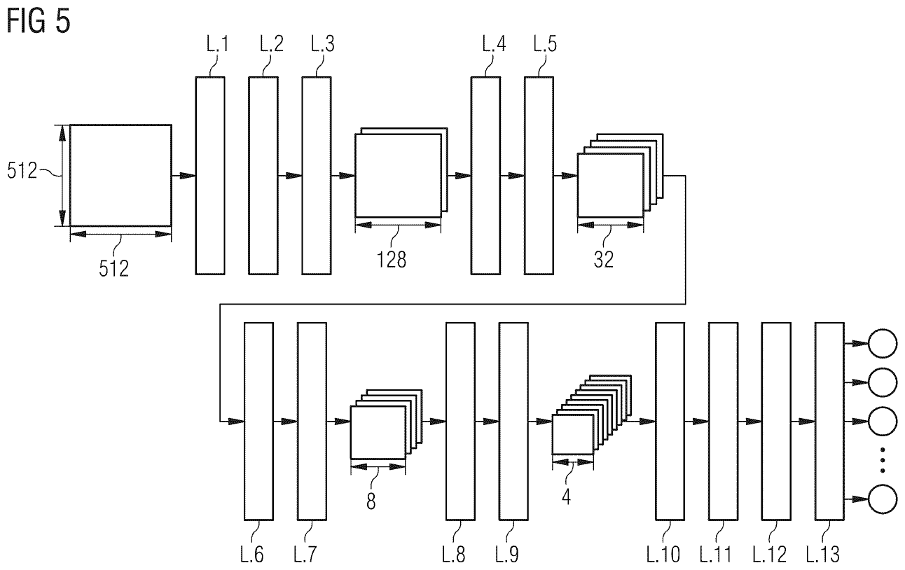

FIG. 6 depicts a medical imaging system. The medical imaging system is adapted to perform the methods according to one or more embodiments, e.g., as further described with reference to FIGS. 1 to 5. A user of the medical imaging system 10, according to some examples, may generally relate to a healthcare professional such as a physician, clinician, technician, radiologist and so forth.

The medical imaging system 10 may comprises a scan unit SU, a computing unit CU and a post-processing unit PPU. Further, the medical imaging system 10 may comprise or be connected to one or more databases (no shown in FIG. 6) generally configured for storing and/or forwarding medical images and/or scan data and/or supplementary information. The components of the medical imaging system 10 may also be referred to as “recipients” as they may receive data such as medical images and information derived therefrom.

The database may for example comprise one or more storage devices for medical images which may be realized in the form of one or more cloud storages, local or spread storage modules, e.g., as a PACS (Picture Archiving and Communication System). The database DB-I may generally be configured for storing and/or forwarding the image data and/or scan data.

The scan unit SU may comprise one or more medical imaging modalities (further details not shown) for acquiring medical images and/or scan data, such as a computed tomography system, a magnetic resonance system, an angiography (or C-arm X-ray) system, a positron-emission tomography system, a mammography system, an X-ray system, or the like.

First scan data and/or subsequent scan data, jointly referred to as scan data, obtained by the scan unit SU may be three-dimensional image data sets, for instance using an X-ray system, a computed tomography system or a magnetic resonance imaging system or other systems. The image data may be encoded in a three-dimensional array of m times n times p voxels. The scan data may include a plurality of image slices which are stacked in a stacking direction to span the image volume covered by the scan data. The scan data may comprise two-dimensional medical image data with the image data being encoded in an array of m times n pixels. According to some examples, these two-dimensional medical images may have been extracted from three-dimensional image data sets.

The medical imaging system 10 may comprise a user interface (not shown). The user interface may comprise a display unit and an input unit. User interface may be embodied by a mobile device such as a smartphone or tablet computer. Further, the user interface may be embodied as a workstation in the form of a desktop PC or laptop. The input unit may be integrated in the display unit, e.g., in the form of a touch screen. As an alternative or in addition to that, the input unit may comprise a keyboard, a mouse or a digital pen and any combination thereof. The display unit may be configured for displaying scan data and or scan geometry data.

The computing unit CU may be a general processor, central processing unit, control processor, graphics processing unit, digital signal processor, three-dimensional rendering processor, image processor, application specific integrated circuit, field programmable gate array, digital circuit, analog circuit, combinations thereof, or other now known devices for processing image data. The computing unit CU may comprise or be connected to a plurality of dedicated repositories or databases. The processor may be single device or multiple devices operating in serial, parallel, or separately. The processor may be a main processor of a computer, such as a laptop or desktop computer, or may be a processor for managing some tasks in a larger system, such as in the medical information system or the server. The processor is configured by instructions, design, hardware, and/or software to perform the steps discussed herein. Further, the computing unit CU may comprise a memory such as a RAM for temporally loading scan data and any intermediate processing results.

The post-processing unit PPU may comprise image processing tools or other post-processing tools and/or functions. Image processing tools may generally be configured to be applied to medical image data, in particular scan data. In other words, these are tools which are configured to process image data in order to provide a corresponding post processing data PPD. The post processing data PPD may be related to image processing result, like medical findings and or segmentations. According to some examples, the post-processing tools may be specialized for a certain use-case such as a type of medical image data (e.g., MR image data or CT image data) and/or a certain type of image processing result. For instance, one of the post-processing tools may be configured to segment and/or detect lesions in an MR scan of a patient, while another post-processing tool may be configured to segment bones in a bone window of a CT scan. The post-processing unit may comprise a post-processing tool database, for example comprising image processing algorithms which are available for processing all kinds of image data which may occur at a certain healthcare facility or diagnostic workplace.

Individual components of medical imaging system 10 may be at least temporarily connected to each other for data transfer and/or exchange. User interfaces may communicate with computing unit CU to exchange, e.g., medical images, elements of a graphical user interface or any user input made. The communication may be realized as hardware- or software-interface, e.g., a PCI-bus, USB or fire-wire. Data transfer may be realized using a network connection. The network may be realized as local area network (LAN), e.g., an intranet or a wide area network (WAN). Network connection is preferably wireless, e.g., as wireless LAN (WLAN or Wi-Fi). Further, the network may comprise a combination of different network examples.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for providing subsequent scan data of a subject, the computer-implemented method comprising:
obtaining, from a medical imaging system, first scan data of the subject;
providing a trained machine learning model configured to determine, based on the first scan data, scan geometry data for a subsequent scan;
applying the trained machine learning model to the first scan data to obtain scan geometry data for a subsequent scan of the subject;
obtaining, from the medical imaging system, the subsequent scan data of the subject using the scan geometry data; and
providing the subsequent scan data.

2. The computer-implemented method according to claim 1, further comprising:
processing the subsequent scan data by a post-processing function, the post-processing function being configured to generate post-processed data based on the subsequent scan data, and wherein
the trained machine learning model is configured such that the scan geometry data is adapted to the post-processing function.

3. The computer-implemented method according to claim 2, further comprising:
determining at least one parameter of the trained machine learning model based on output data of the post-processing function.

4. The computer-implemented method according to claim 3,
wherein the trained machine learning model has been trained using a loss function based on the post-processed data.

5. The computer-implemented method according to claim 1,
wherein the first scan data includes image data from an initial scan,
wherein the subsequent scan data includes image data from a subsequent scan,
wherein the initial scan is obtained based on initial scan parameters,
wherein the subsequent scan is obtained based on subsequent scan parameters, and
wherein the subsequent scan parameters are determined based on the scan geometry data.

6. The computer-implemented method according to claim 1,
wherein an initial scan to obtain the first scan data and a subsequent scan to obtain the subsequent scan data differ in at least one of resolution parameters, scan geometry, imaging area or region of interest.

7. The computer-implemented method according to claim 1,
wherein scan geometry data includes an align-matrix,
wherein the first scan data includes scan parameters, and
wherein the align-matrix includes alignment information for obtaining the subsequent scan data with respect to the scan parameters.

8. The computer-implemented method according to claim 1, further comprising:
reporting the scan geometry data to a user via a user interface.

9. The computer-implemented method according to claim 1, further comprising:
receiving user input to provide additional scan geometry data via a user interface; and wherein
obtaining the subsequent scan data is additionally based on the additional scan geometry data.

10. A computer-implemented method for providing a trained machine learning model, the computer-implemented method comprising:
receiving input training data including first scan training data, subsequent scan training data, and post-processing training data;
predicting scan geometry data by applying the trained machine learning model to the first scan training data;
obtaining aligned subsequent scan data by applying the scan geometry data to the subsequent scan training data to simulate a scan;
obtaining post-processing data by inputting the aligned subsequent scan data into a post-processing function, the post-processing function being configured to generate post-processed data based on the aligned subsequent scan data;
adapting the trained machine learning model based on a comparison of the post-processing data and the post-processing training data; and
providing the trained machine learning model.

11. The computer-implemented method according to claim 10, wherein obtaining the aligned subsequent scan data comprises:
planning a scan direction in an isotropic volume of the subsequent scan training data based on the scan geometry data; and
simulating a subsequent scan based on at least one of the scan geometry data or the scan direction to receive an anisotropic volume slicing.

12. The computer-implemented method according to claim 10,
wherein the post-processing function includes one or more parameters, and wherein
the one or more parameters are adapted during training of the trained machine learning model.

13. The computer-implemented method according to claim 1, wherein the trained machine learning model is provided by a method including:
receiving input training data including first scan training data, subsequent scan training data, and post-processing training data;
predicting scan geometry data by applying the trained machine learning model to the first scan training data;
obtaining aligned subsequent scan data by applying the scan geometry data to the subsequent scan training data to simulate a scan;
obtaining post-processing data by inputting the aligned subsequent scan data into a post-processing function, the post-processing function being configured to generate post-processed data based on the aligned subsequent scan data;
adapting the trained machine learning model based on a comparison of the post-processing data and the post-processing training data; and
providing the trained machine learning model.

14. A medical imaging system, comprising:
a scan unit configured to acquire first scan data of a subject;
a computing unit configured to obtain scan geometry data from the first scan data with a trained machine learning model, wherein
the scan unit is further configured to acquire subsequent scan data of the subject based on the scan geometry data; and
a post-processing unit configured to output post-processed data from the subsequent scan data with a post-processing function, wherein
the trained machine learning model is configured to output scan geometry data tailored for the post-processing function.

15. A non-transitory computer program product comprising program elements that induce a computing unit to perform the computer-implemented method according to claim 1 when the program elements are loaded into a memory of the computing unit.

16. A non-transitory computer-readable medium on which program elements are stored, the program elements being readable and executable by a computing unit to perform the computer-implemented method according to claim 1 when the program elements are executed by the computing unit.

17. The computer-implemented method of claim 1, wherein the scan geometry data includes slice alignment data.

18. The computer-implemented method according to claim 2,
wherein the first scan data includes image data from an initial scan,
wherein the subsequent scan data includes image data from a subsequent scan,
wherein the initial scan is obtained based on initial scan parameters,
wherein the subsequent scan is obtained based on subsequent scan parameters, and
wherein the subsequent scan parameters are determined based on the scan geometry data.

19. The computer-implemented method according to claim 2,
wherein scan geometry data includes an align-matrix,
wherein the first scan data includes scan parameters, and
wherein the align-matrix includes alignment information for obtaining the subsequent scan data with respect to the scan parameters.

20. The computer-implemented method according to claim 5,
wherein scan geometry data includes an align-matrix,
wherein the first scan data includes scan parameters, and
wherein the align-matrix includes alignment information for obtaining the subsequent scan data with respect to the initial scan parameters.
